(19) United States
(12) Patent Application Publication
(10) Pub. No.: US 2005/0251095 A1
(43) Pub. Date: Nov. 10, 2005

(54) IMPROVED MEDICATION INJECTION DEVICE

(76) Inventor: Brandon C. Giap, San Diego, CA (US)

Correspondence Address: LAW OFFICE OF MICHAEL P. EDDY, 12526 HIGH BLUFF DRIVE, STE. 300, SAN DIEGO, CA, 92130, US

(21) Appl. No.: 10/908,019

(22) Filed: Apr. 25, 2005

Related U.S. Application Data
(60) Provisional application No. 60/569,425, filed on May 7, 2004.
(60) Provisional application No. 60/594,631, filed on Apr. 25, 2005.

(51) Int. Cl. A61M 5/00

(52) U.S. Cl. 604/181

(57) ABSTRACT

An injection device made up of a housing consisting of a base and a top, a deformable reservoir containing medication in connection with the base ends of the needles, the plurality of needles contained in said housing base, and a gel layer disposed on the plurality of needles, wherein each said needle has a base end and a tip and a hollow pathway disposed at or between each base end and the tip and wherein said medication is injected into a patient through said plurality of needles when said housing top is depressed into said housing base.

### CROSS-REFERENCES TO OTHER RELATED PATENT APPLICATIONS

This application claims priority to U.S. provisional application Ser. No. 60/569,425 filed May 7, 2004 and U.S. provisional patent application Ser. No. 60/594,631, filed Apr. 25, 2005, both of which are incorporated herein by reference in their entireties.

### BACKGROUND

The present invention relates to a novel device for injecting medication into a patient.

Delivering medication to patients using syringes and needles is an everyday task for medical professionals. For example, doctors or nurses anesthetize the skin area with local anesthetic using a small needle prior to starting an intravenous catheter.

However, often even a small needle produces an appreciable amount of discomfort to the patient. This discomfort may be in part real and in part psychological. The physical/real pain is caused by the intrusion of the needle, notwithstanding its small size and length. Yet another contributing factor to the pain is expansion of the charged tissue with the anesthetic or medicine. The psychological component, however, is associated with the patient's intolerance to blood or to needles, with such intolerance often regardless to the diameter or the length of the needle being used.

A common situation where there is discomfort associated with using needles is the use of needles with children. Such discomfort can be mitigated, but can be more problematic as the child moves about and cries. This scenario evokes a negative response in the staff, raises apprehension in other children or patients, and it is very disturbing and worrisome of the parents. If the negative response is known ahead of time to the doctor, he or she may choose to apply an oral sedative. This has its own downside, as the oral sedative may be vomited and aspirated causing further complications.

Thus, there remains a need for a medication delivery vehicle that eliminates both the components of the physical/real pain and psychological pain.

### SUMMARY

The disclosed, novel device employs multiple needles in conjunction with a reservoir of medicine that is applied through the skin when the medicine is pushed through the needles with a mechanical force, such as when done with a human finger or hand. The plurality of needles over an area of skin or tissue limits the intrusion of any one needle and is relatively painless because less tissue is penetrated or disturbed by each of such needles. In effect, an array of small needles used within the device would have a similar pain factor to pressing abrasive sand paper against the skin. Moreover, the medicine delivered by each of the plurality of needles is a fraction of the volume that is delivered by a single needle application and therefore the tissue is not stretched as much as it would be by a single needle application.

Embodiments can also address the psychological pain factor because the needles are small and the device does not look like a conventional syringe and needle combination. The device can be shaped and configured as a “button,” a “fish” or any other shape to minimize the apprehension and to divert or eliminate the unnecessary and harmful factors of fear and discomfort.

Other methods, features and advantages of the invention will be or will become apparent to one with skill in the art upon examination of the following figures and detailed description. It is intended that all such additional systems, methods, features and advantage be included within this description, be within the scope of the invention, and protected by the accompanying claims.

### BRIEF DESCRIPTION OF THE DRAWINGS

FIG. 1 is a perspective view of an embodiment of the claimed subject matter;

FIG. 2 is another perspective view of an embodiment of the claimed subject matter;

FIG. 3 is a side view of an embodiment of the claimed subject matter showing the cover positioned over the gel layer;

FIG. 4 is a side view of an embodiment of the claimed subject matter showing the cover removed;

FIG. 5 is a side perspective view of an embodiment of the claimed subject matter showing the cover removed;

FIG. 6 is top view of an embodiment of the claimed subject matter; and

FIG. 7 is a side cross-sectional view of an embodiment of the claimed subject matter.

### DETAILED DESCRIPTION OF THE EMBODIMENTS

Embodiments of the claimed subject matter relate to an improved medication injection device. A description of various exemplary aspects of embodiments of the claimed subject matter will now be provided.

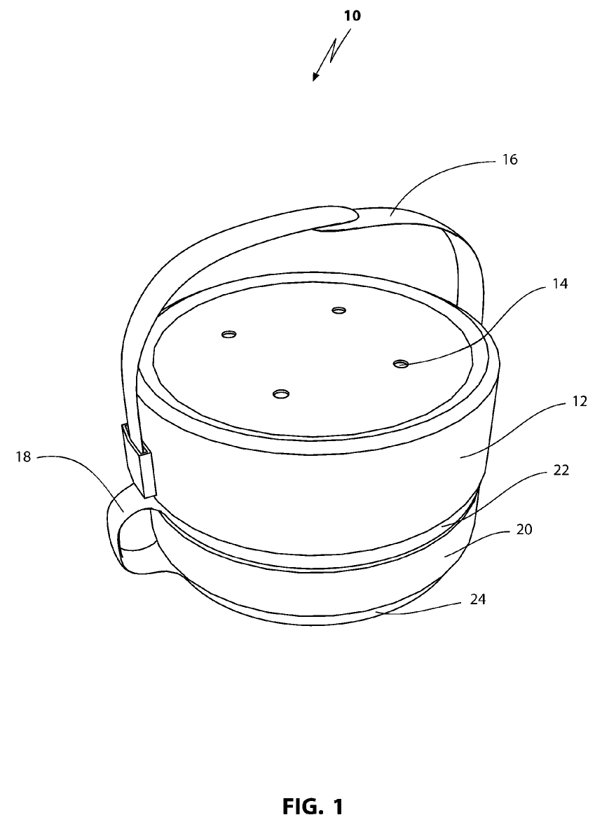

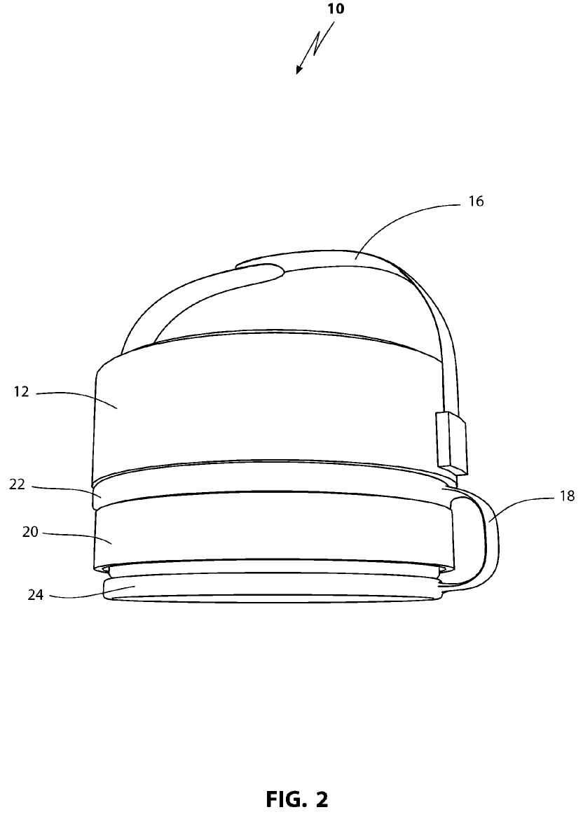

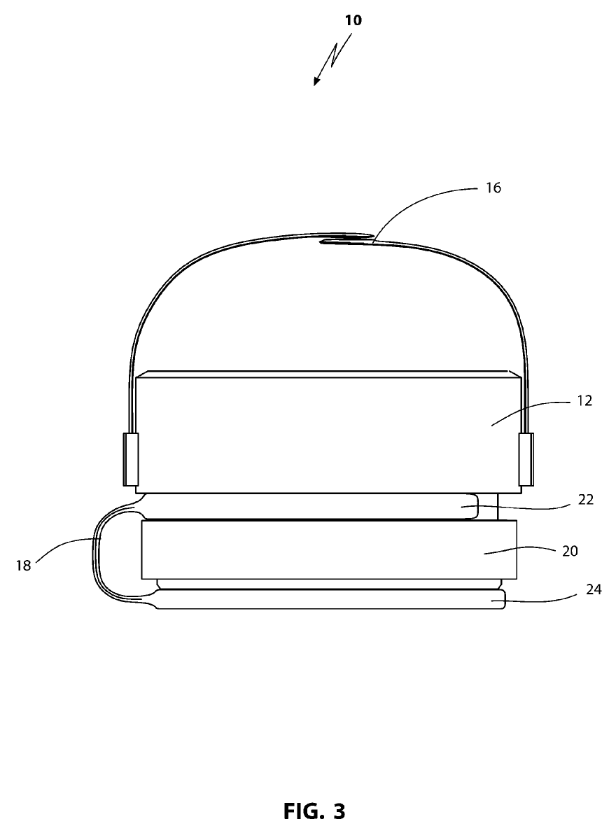

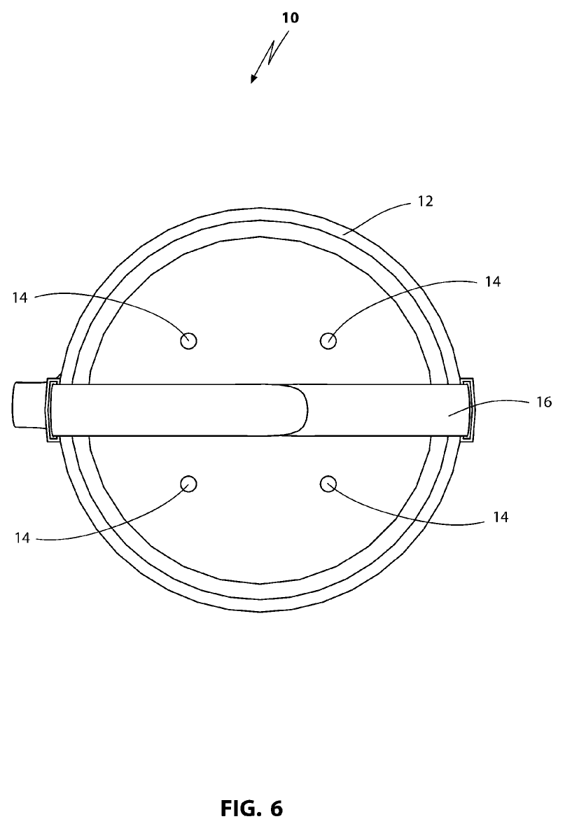

An embodiment of the claimed subject matter is illustrated in FIGS. 1 through 7. In this embodiment, housing top 12 and housing base 20 are disposed in a respective, movable relationship, thus allowing the housing top 12 to move in relation to housing base 20 and the needles 28 attached thereto. As the housing top 12 moves in relation with the housing base 20, the medicine contained in reservoir 30 is forced through needles 28 and released into to the target tissue.

The housing top 12 is illustrated as round with vent holes to allow the top to be depressed into the bottom housing. The housing top 12 and housing base 20 are complementary and may be square, rectangular or any other suitable shape or configuration. The housing top 12 is slidably depressed over the housing base 20 in order to create mechanical pressure on the reservoir 30. The device 10 is activated by depression with a hand or one or more human fingers. There are four vent holes 14 illustrated on the housing top 12. As the exterior surface of housing top 12 is depressed, it forces the medicine, such as a local anesthetic, contained in reservoir 30 through the plurality of needles 28 into the patient.

The housing top 12, housing base 20, and substrate 32 may be constructed from a variety of materials, including plastic metals, ceramics, semiconductors, organics, polymers, and composites, and device 10 may have the diameter of an average button, or about 1 cm. The device 10 can also be larger or smaller in diameter. In another embodiment, the shape of the housing top 12 may be a dome so that when housing top 12 is depressed, the housing top 12 will more easily release the local anesthetic via the needles into the skin. In a pediatric setting, the housing top 12 may also accommodate a dispenser for removable stickers or labels to alleviate anxiety in the younger patients.

In other embodiments of the device 10, the substrate 32, housing top 12 and housing base 20 may be formed from flexible materials to allow the device 10 to fit the contours of the biological barrier, such as the patient's skin. In other embodiments the substrate 32, housing top 12 and housing base 20 are formed from rigid materials.

Housing top 12 slides over base 20 when device 10 is depressed so that medicine is delivered from the array of needles 28 protruding from housing base 20. Further, some embodiments may use a clear material for housing top 12 so that an administrator can easily determine whether or not a device 10 has been discharged. In another embodiment, the housing top 12 is configured in the shape of a dome top, which is made out of an elastic plastic or similar material, that gives the housing 12 an elastic quality, similar to a pop-up top when one open a top of a glass jar. When an adequate force is applied to the patient's skin and the needles have entered through the patient's skin, the convex top “pops” downward into a concave position rapidly pushing the reservoir 32 and liquid medication into the patient's tissue.

Reservoir 30 is in connection with each needle bore, such that the reservoir contents can flow from the reservoir and out through the needle tips, into the target tissue. Reservoir 30 along with the needles 28 are integrated into the housing base 20. Reservoir 30 provides leak-free storage of a medicine before it is to be delivered, and the interior lining of reservoir 30 may be made out of an inert material to give the delivery agent an acceptable shelf life.

The reservoir 30 can be substantially rigid or readily deformable and it can be formed from one or more polymers, metals, ceramics, or combinations thereof. Reservoir 30 is constructed in this way to deform when the housing top 12 is depressed. In one embodiment, the reservoir 30 includes a volume surrounded by one or more walls, or includes a porous material, such as a sponge, which can retain, for example, the drug liquid until the material is compressed. In an embodiment, reservoir 30 is formed of an elastic material, such as an elastomeric polymer or rubber. For instance, reservoir 30 may be a balloon-like pouch that is filled with medicine.

Reservoir 30 may also be comprised of a plurality of compartments that are isolated from one another and/or from one or more needles 28 in an array. In this way, embodiments of the device 10 may provide different medicine through different individual needles that are found in needle array 28. In other embodiments, there is more than one compartment or sub-reservoirs that make up reservoir 30 and each of these compartments may contain a unique type of agent or medicine to be delivered.

Any medicine or drug may be delivered with embodiments of the claimed subject matter. Examples of suitable therapeutic and/or prophylactic active agents include proteins, such as hormones, antigens, and growth factors; nucleic acids, such as antisense molecules; and smaller molecules, such as antibiotics, steroids, decongestants, neuroactive agents, anesthetics, and sedatives. Examples of suitable diagnostic agents include radioactive isotopes and radioopaque agents, metals, gases, labels including chromatographic, fluorescent or enzymatic labels. The drug can be or include a peptide, protein, carbohydrate (including monosaccharides, oligosaccharides, and polysaccharides), nucleoprotein, mucoprotein, lipoprotein, glycoprotein, nucleic acid molecules (including any form of DNA such as cDNA, RNA, or a fragment thereof, oligonucleotides, and genes), nucleotide, nucleoside, lipid, biologically active organic or inorganic molecules, or combination thereof. The amount of drug can be selected by one of skill in the art, based, for example on the particular drug, the desired effect of the drug at the planned release levels, and the time span over which the drug should be released.

Examples of medicine that may be used include Versed, which may be used in pediatric patient populations prior to surgery where I.V. access was not available, or where a child refuses oral administration. Immunization agents may also be used in other embodiments. Other embodiments that may be useful include the use of anesthetic, anti-psychotic, or anti-nausea medications.

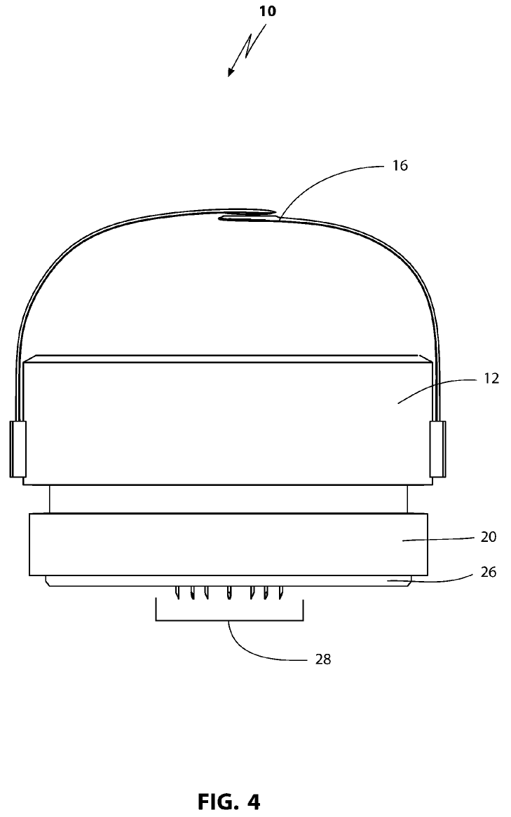

As shown in FIGS. 4, 5 and 7, an embodiment includes a gel layer 26 which covers needles 28 of device 10. This gel layer 26 may substantially cover the needles 28 but leave the tips of the needles 28 exposed on the underside of the device 10 or the layer 26 may completely cover needles 28. This gel layer 26 may also cover all or part of the underside surface of housing base 20. The gel layer 26 can be formed of an antibiotic gel, a cleansing agent, a soothing agent, a cooling agent, a sterilizing agent or the like. The gel layer 26, besides substantially concealing the needles 28 to the patient, brings medicinal, anesthetic, distracting, cleansing and/or otherwise soothing qualities to the patient. Because the needles 28 are covered, they may be unseen by the patient lessening the apprehension and psychological factors as previously described. The gel layer 26 may also include an anesthetic to further dull pain on the skin or an antibiotic to counteract bacterial infection.

A plurality of needles 28 are positioned and integrated into the housing base 20 and a reservoir 30 is in fluid connection with the needles 28. Needles 28 may be placed in an array or in a row or in any other suitable configuration and the number of needles 28 may be varied as needed. Each needle is hollow having a channel with a diameter sufficient to permit passage of a drug-containing fluid and/or other substances through the needle. The hollow needle shafts may be linear or they may be curved. The needles 28 can be constructed from a variety of materials, including metals, ceramics, semiconductors, organics, polymers, and composites. Suitable metals include stainless steel, gold, titanium, nickel, iron, tin, chromium, copper, palladium, platinum, alloys of these or other metals, silicon, and silicon dioxide.

The needles 28 should have the mechanical strength to remain intact while being both inserted into and removed from the biological barrier. Cross-sections of each needle may be polygonal (e.g. star-shaped, square, triangular), oblong, or any other suitable shape. The size and length of the protruding needle tips as well as the spacing between the needles in the housing base 20 may be determined and placed so as to achieve maximal flow of medication with the least amount of discomfort.

In embodiments of the claimed subject matter, other than a small portion of the needle tips that protrude through the gel layer at the base of the housing, the plurality of needles 28 cannot be seen by the patient. Embodiments may also include a plurality of needles 28, with one ore more needles of a different length so that the needles 28 will enter the skin at different times causing less discomfort.

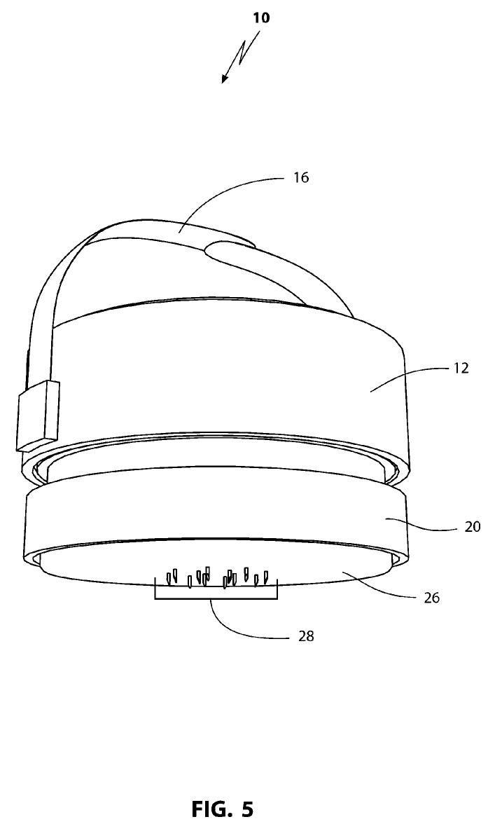

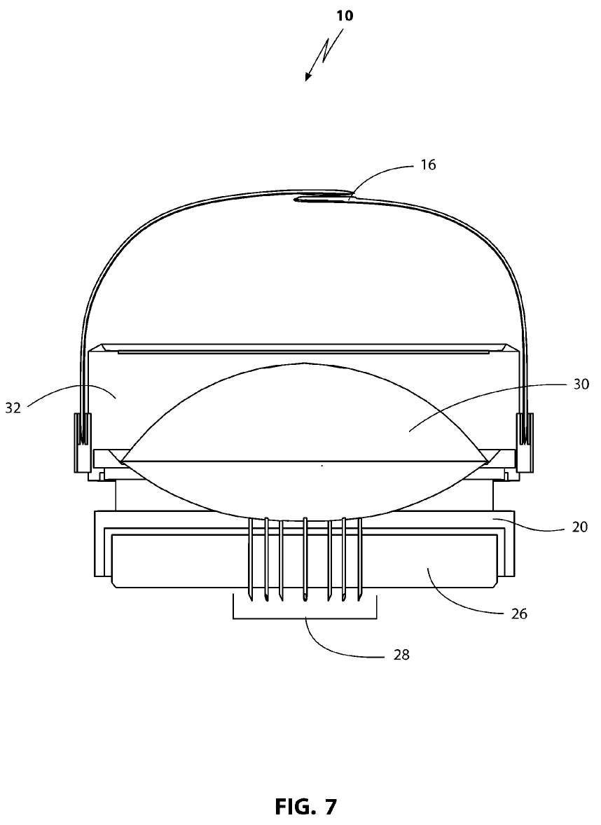

FIGS. 4 and 5 show the device 10 with needles 28 protruding from the housing base 20. In the illustrated embodiment, the varying length needles 28 are integrally formed into housing base 20. FIG. 7 shows an array of needles 28 in varying lengths. Other embodiments can include a mixture of needles having varying lengths, outer diameters, inner diameters, cross-sectional shapes, and space between the needles.

The diameter of the needles used with the device can be any commercially available size, so long as the needles can penetrate the skin and reach the desired depth. The length of each needle will vary depending on the application and design of the device, and each needle will have a portion that is inserted into the patient's skin and a portion that remains external to the patient's skin.

If an intradermal layer injection is desired, for example using a local anesthetic, a TB skin test, or an allergy skin test, the protruding needle length can be less than 1 mm, or even shorter if being used in children. If a subcutaneous layer injection is desired (under the skin,) the needle length may be longer that 1 mm, perhaps between 1 mm to 2 mm. The needles can also be made long enough to achieve intramuscular injection. Although aspiration is not possible with all embodiments of the claimed subject matter, the needles 28 would not be stationary during the injection procedure, so the plurality of needles 28 would each reach a different area under the skin.

In the case of a tuberculosis (TB) skin test or an allergy test, the length of the protruding needles should all be consistent so that the medication is delivered precisely at a certain depth into the skin in order to facilitate an accurate follow up reading at a later time.

As illustrated in FIGS. 1 through 7, a finger strap 16 may be attached the side of the housing top 12 to assist in the handling of the device 10 including the placement and discharge of device 10. The strap 16 may be a hand strap, finger strap or the like. It would allow an administrator of the medicine to keep from touching the sterile underside of the device 10, to give the device 10 a more precise placement on the skin, and to prevent accidental or unintended discharge of medicine during preparation of device 10.

The finger strap 16 that extends from both sides of the housing top 12 can be two sections with ends that are attached with hook and loop material such as Velcro. Strap 16 may be of different shapes, configurations and sized to accommodate varying uses. For instance, a large strap 16 may be used for a hand or for larger device 10. In one embodiment, strap 16 wraps around an administrator's finger, preferable distal ⅓ of the index or longer finger. This configuration will allow the device stay securely on that person's finger prior to applying onto the subject patient.

The pull tab 18 and safety C-ring 22 are removed before the housing top 12 can be depressed into the housing base 20. Pull tab 18 is connected to the safety C-ring 22 which is used to prevent the device 10 from accidental discharge. C-ring 22 may be easily removed with pull tab 18 just prior to the application of the device onto a patient's skin after the device 10 has been secured on the administrator's finger or hand. After the injection, the device 10 can be easily slipped off the hand or finger and safely discarded.

In the embodiment, a removable protective cover 24 protects the gel layer 26 and needles 28 and serves to keep the gel layer 26 as well as the needles 28 in a sterile environment. The interior of the protective cover 24 may include antiseptic materials. After the device 10 has been discharged, the cover can be replaced over the gel layer and needles 28 for safe disposal of the used device 10. The safety C-Ring 22 may also be replaced to prevent reuse of the device 10.

In another embodiment, the exterior of the removable protective cover 24 can hold an antiseptic wipe to prepare the skin surface before insertion of the needles 28. Cover 24 may also hold a dispenser for removable adhesive stickers that can be peeled off and given to the patient before the device 10 is used. The administrator may also swab the patient's skin down with a separate antiseptic swab prior to use of the device 10.

In use, delivery of the medicine from the reservoir 30 is initiated by applying a force, such as by pressing the housing top 12, to cause the reservoir 30 contents to flow out through the needles into the skin. In this example, the user applies his or her finger-pressure directly to the housing top 12 which deforms reservoir 30 forcing the medicine from the reservoir through the needles 28 and into the patient's skin. Another embodiment utilizes the elastic properties of reservoir 30 to allow the reservoir 30 to contract to its natural state once it has been triggered by finger pressure. The contraction of the reservoir then provides the driving force for delivery of the medicine.

Additionally, as the device 10 is applied to patient, the gel layer 26 is compressed allowing the needles 28 to enter the skin while the medication is being released. This gel layer 26 helps lessen the roughness of the needle surface and reduces discomfort in the patient. Additionally, if the device 10 is kept in the refrigerator, the gel layer 26 would be cool on contact with the surface of the patient's skin and this may further lessen the discomfort of the needles piercing the skin.

Embodiments of the device 10 may be used for applying local anesthetic prior to inserting a needle for intravenous (IV's) catheter insertion or blood drawing, and the disclosed device can be prefilled or pre-charged by the pharmaceutical supplier for a single use or filled and refilled by the user or other person with a syringe through a port or gasket (not shown) located on the housing top 12 before use. The device may also be used without pre-filled medication. Embodiments of the device may single-use or they may be reusable with a port on the top for injecting with a syringe a supply of medication.

Other embodiments of the claimed subject matter include the use of epinephrine for emergency situations such as when used before attempting to establish an intravenous line. An embodiment prefilled with epinephrine can also be useful in patients with moderate to severe asthma who are unable to use an inhaler or bronchodilator such as albuterol. Another embodiment could be prefilled with atropine so it could be also be used during a cardiac arrest event. Other embodiments could be used to deliver anti nerve gas agents for soldiers in battle or epinephrine for bee sting attacks. Embodiments with larger needles 28 can be used for security and law enforcement purposes wherein the needles 28 can be used to pierce clothing in emergency situations.

Similar methods and structures could be employed to achieve the goals of the present invention. Those of ordinary skill in the art will recognize that many more embodiments, equivalents and implementations are possible that are within the scope of this invention. The described embodiments are provided by way of example and are not intended to limit the invention.

1. An injection device comprising:
a housing consisting of a base and a top disposed in a respective, movable relationship so as to allow the housing top to slidably move in relation to housing base;
a plurality of needles integral to and protruding from said housing base;
a deformable reservoir containing medication in connection with the base ends of said plurality of needles; and
a gel layer disposed over said plurality of needles;
wherein each said needle has a base end and a tip and a hollow pathway disposed at or between each base end and the tip and wherein said medication is injected into a patient through said plurality of needles when said housing top is depressed into said housing base.

2. An injection device of claim 1, further comprising a safety C-ring to prevent accidental discharge of said device.

3. An injection device of claim 1, further comprising a finger tab to hold the device to a user's finger.

4. An injection device of claim 1, further comprising a strap to hold the device to a user's hand.

5. An injection device of claim 1, further comprising a removable protective cover for covering said gel layer and needle tips.

6. An injection device of claim 1 wherein the reservoir is constructed from an elastic material.

7. An injection device of claim 1 wherein the reservoir is formed of a material which is deformable.

8. An injection device of claim 1 wherein the medicine stored in the reservoir comprises a therapeutic, prophylactic, or diagnostic agent.

9. An injection device of claim 1 wherein the medicine is selected from the group consisting of peptides, proteins, carbohydrates, nucleic acid molecules, lipids, organic molecules, biologically active inorganic molecules, and combinations thereof.

10. An injection device of claim 1 wherein the diameter of the needles is between about 1 micrometers and about 100 micrometers.

11. An injection device of claim 1 further comprising a hand strap.

12. An injection device of claim 1 further comprising a port on the housing top for refilling the device with a syringe.

13. An injection device of claim 1 further comprising a removable protective cover for the gel layer and needles.

14. An injection device of claim 1 wherein the reservoir is made up of multiple compartments.

15. An injection device of claim 5, wherein said cover accommodates an external antiseptic swap.

16. An injection device of claim 1, further comprising a port for refilling the reservoir.

17. An injection device of claim 1 wherein at least one of the needles is of a different length than the others.

18. An injection device of claim 1 wherein the housing top, housing base and substrate are constructed from a flexible material.

19. An injection device of claim 1 wherein the gel layer includes an anesthetic.

20. An injection device of claim 1 wherein the gel layer includes an antibiotic.
